(19) United States
(12) Patent Application Publication
(10) Pub. No.: US 2008/0216825 A1
(43) Pub. Date: Sep. 11, 2008

(54) Insulated Cansister for Metered Dose Inhalers

(76) Inventors: Dilraj Singh, Basel (CH); Gerard Provot, Evreux (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/996,981

(22) PCT Filed: Aug. 4, 2006
(86) PCT No.: PCT/EP06/65095

§ 371 Date: Jan. 28, 2008

Related U.S. Application Data
(60) Provisional application No. 60/706,495, filed on Aug. 8, 2005.

(51) Int. Cl. A61M 11/00

(52) U.S. Cl. 128/200.23

(57) ABSTRACT

An insulated canister for, pressurized or non-pressurized systems, use with a metered dose system for example, a metered dose inhaler or topical aerosol featuring an inner container surrounded by an outer container with a gap defined by the space between the walls of the inner and outer container. Such gap can be filled by a vacuum, air or material with low thermal conductivity.

### FIELD OF THE INVENTION

The present invention relates to an insulated canister for use in a metered-dose delivery system, for example an inhaler. In particular, the present invention features a canister having a dual wall.

### BACKGROUND OF THE INVENTION

Therapeutic compounds for treating respiratory, nasal and skin diseases and conditions are often formulated into aerosol formulations for delivery via oral, nasal or topical routes of administration. The therapeutic compounds are supplied in the form of a suspension or a solution, that is to say the therapeutic compound is present in a container, for example under pressure or not (e.g., nasal aqueous inhalers), in the form of small solid particles suspended or dissolved in a vehicle. For a pressurized system, such a system can be a liquefied gas known as a propellant. When sealed, the container is capable of withstanding the pressure required to keep the gas liquefied. The suspension or solution is administered through a metering valve that releases a fixed and constant amount of medication upon each use. Once expelled through the metering valve, the propellant quickly vaporizes releasing the therapeutic compound to be inhaled or deposited on the skin. The delivery of the therapeutic compound is guided to the mouth and/or nasal passages or skin of the user by an adapter. Such delivery devices are known as “metered dose inhalers” (MDIs) when used for releasing either an inhaled therapeutic compound or a “topical aerosol” when administering a topically applied therapeutic compound.

With the advent of high throughput screening in research, new therapeutic compounds are often being identified for their biological and pharmacological activity. However, a therapeutic compound's activity does not ensure success during development and commercialization. A common reason for this lack of developability is the physical characteristics of the therapeutic compound. For example, the poor water solubility of a therapeutic compound can render that compound not bioavailable when administered. Another possible characteristic that can impact the viability of the therapeutic compound is the chemical stability at varying temperatures. A therapeutic compound may degrade, either chemically or physically, when exposed to temperatures at or greater than room temperature. Such a thermally labile therapeutic compound would not be capable of being delivered via conventional MDIs that are stored and used at room temperature. Thus, there is a need for a MDI with an insulated canister that can maintain the canister and its contents at a temperature that minimizes thermal degradation. Furthermore, there is a need for a MDI with an insulated canister that minimizes the impact of the temperature on the contents within the canister. The present invention addresses such needs.

### SUMMARY OF THE INVENTION

The present invention relates to a dual wall canister that is suitable for use with a metered dose delivery system, for example a metered dose inhaler. The canister has an outer container and inner container both shaped such that it fits within the outer container. Defined between the walls of the outer container and the inner container is a gap that can be filled with a vacuum or a material of low thermal conductivity.

In another embodiment of the present invention is a drug delivery system, for example a metered dose delivery system that features a pharmaceutical composition in an insulated dual wall canister. The dual wall canister has an inner container disposed within an outer container such that a gap is defined by the space between the inner and outer containers. The pharmaceutical composition can, for example, contain a therapeutic compound, especially one that is thermally labile and suitable for inhalation or topical administration. Particularly suited therapeutic compounds are those for respiratory and dermatology indications, diseases and conditions.

These and other features, advantages and objects of the present invention will be further understood and appreciated by those skilled in the art by references to the following specification, claims and appended drawings.

### BRIEF DESCRIPTION OF THE DRAWINGS

The accompanying drawings, which are incorporated in and constitute a part of the specification, illustrate an exemplary embodiment of the present invention.

FIG. 1 shows a cross-sectional view of an insulated canister drinking vessel shown in its “in use” position in accordance with an exemplary embodiment of the present invention.

### DETAILED DESCRIPTION OF THE INVENTION

The present invention features an insulated canister appropriate for use as a storage container of a pharmaceutical composition that includes a thermally labile therapeutic compound. The insulated canister, for example, can be used as a component of a MDI, a topical aerosol canister, or a nasal aqueous inhaler. The insulated canister includes a dual walled structure that has an outer container encompassing an inner container with a different taper than the outer container to form an insulating gap between the outer and inner containers. The inner container defines a cavity 32 that holds the pharmaceutical composition (as hereinafter defined) to be administered by the delivery system. An insulating material, as defined below, is disposed within the insulating gap. Furthermore, the interior surface of the inner container can be optionally coated with a coating substrate material.

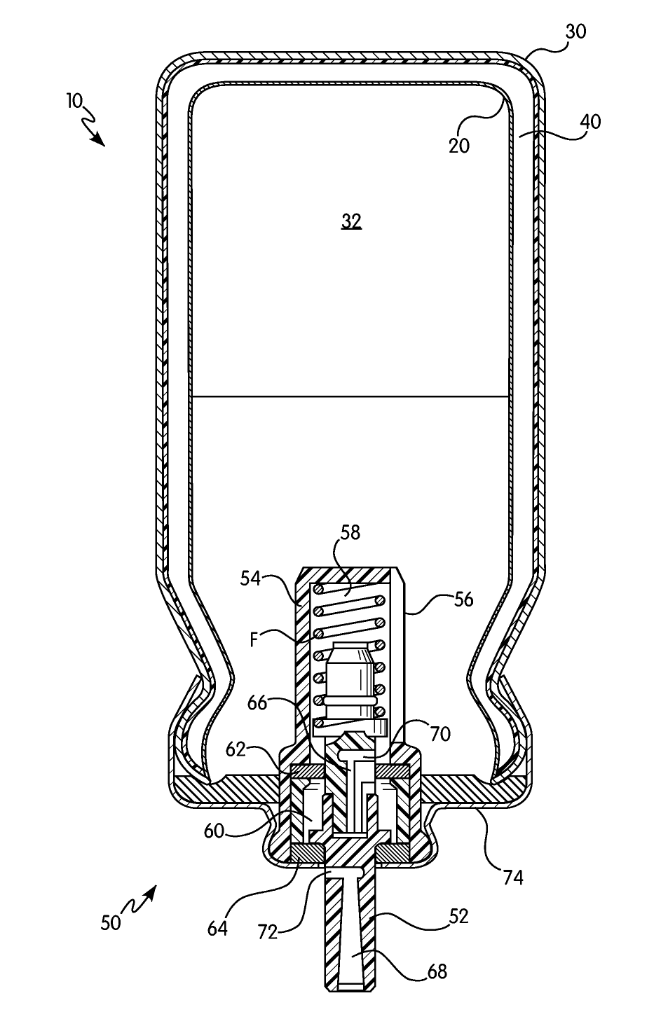

FIG. 1. shows a cross-sectional view of an exemplary embodiment of an insulated canister 10 for a pressurized pharmaceutical composition, in accordance with the present invention. The insulated canister 10 is comprised of an outer container 20 and an inner container 30. Containers 20 and 30 are inserted within each other, and as a result, a gap 40 is created therebetween. Each of the containers 20 and 30 have a side wall and a bottom wall. The walls can each have a thickness in the range from about 0.1 mm to about 2 mm, e.g., 0.4 mm. As used herein the term “about” includes the values disclosed and variations thereof within engineering tolerances.

The containers 20 and 30 can be made from a material known from the prior art to be suitable for use as canister materials in the pharmaceutical industry, for example pure metals and metal alloys. Such metal or metal alloys can be optionally pre-treated or processed, e.g., galvanized, annealed and/or plated. Examples of metals include, but are not limited to, aluminum, steel, copper, brass, tin and chromium.

Optionally coated along the inside surface of the inner container 30 is a surface coating 34. The surface coating 34 can be made from a material known from the prior art to be compatible with pharmaceutical compositions contained within MDIs and topical aerosols. Examples of suitable surface coatings 34 include, include but are not limited to coatings of a fluorocarbon polymer, e.g., polytetrafluoroethylene, ethylenetetrafluoroethylene, polyinyidienefluoride, perfluoroalkoxyalkane, polyvinylfluoride, polychlorotrifluoroethylene and fluorinated ethylenepropylene; an epoxy-phenol resin; and glass. Particularly useful as coatings 34 are those fluorocarbon polymers that have a relatively high ratio of fluorine to carbon, such as perfluorocarbon polymers, e.g., polytetrafluoroethylene, perfluoroalkoxyalkane and fluorinated ethylenepropylene. The use of these materials prevents significant deposits of the therapeutic compound on the inside surface of the inner container 30. The effects of corrosion and electrolysis between the inner container and the pharmaceutical composition are avoided.

A wide variety of processes may be used to produce the surface coating 34 on the inside surface of the inner container 30. For example, the coating process used may be plasma coating, an impregnating/spraying process, hard anodization with PTFE inclusion, chemical vapor deposition, physical vapor deposition and other process that are customary for that purpose. Particularly useful is plasma coating. The coating thickness, for example, can be in the range of about 0.1 micron to about one millimeter, e.g., one to a hundred microns, e.g., one to twenty-five microns.

The gap 40 between containers 20 and 30 is closed, and thus reduces the heat transfer between the contents of the canister 10 and the surrounding environment. In an exemplary embodiment of the present invention the gap 40 is filled with a gas, for example air or nitrogen. The gas can also be a low thermoconductive gas, for example, xenon, krypton and argon.

In an alternative exemplary embodiment, the gap consists of a negative pressure, i.e. a vacuum. As used herein the term “negative pressure” refers to any pressure less than atmospheric pressure up to a perfect vacuum. For example, the negative pressure may be in the range of about 400 mbars to about 800 mbars, e.g., from about 500 mbars to about 700 mbars.

Alternatively, the gap 40 can be occupied by a material with a low thermal conductivity. As used herein, the term “thermal conductivity” refers to a material's ability to transfer heat via conduction. The thermal conductivity for an appropriate material, for example, can range from about 0.0001 to 0.5 W·m⁻¹·K⁻¹. Examples of materials with low thermal conductivity in addition to the ones previously mentioned, include, but are not limited, to foams, e.g., made from celluloid, nylon, polystyrene polyethylene terphthalate, and polyurethane; aerogels, wools, e.g., mineral, cotton and steel; refractory materials, e.g., zirconium oxide, aluminum oxide and rubber.

Mounted on the canister 10 is a metering valve 50. The metering valve 50, for example, includes a valve stem 52, which is guided in a valve housing 54, and is displaceable against the force of a spring F in the valve housing 54. Provided in the wall of the valve housing 54 are individual slots 56 which place the cavity 32 of the inner container 30 in communication with the interior 58 of the valve housing 54. The metering valve 50 also comprises a metering chamber 60, which is filled, as explained below, through the slots 56 in the wall of the valve housing 54 with the aid of the valve stem 52. The interior 58 of the valve housing 54 is sealed from the metering chamber 60 by means of a metering gasket 62; the metering chamber 60 is in turn sealed from the outside by a stem gasket 64. Finally, the entire cavity 32 of the inner container 30 is in addition sealed by means of a sealing gasket 74 provided in the metering valve 50.

The valve stem 52 of the metering valve 50 has two channels, a first channel 66 and a second channel 68. The first channel 66 has at its “inner” end a first transverse bore 70 which, in the illustrated first position of the valve stem 52, opens into the interior 58 of the valve housing 54 and thus places the interior 58 of the valve housing 54, and therefore the cavity 32 of the canister 10, in communication with the metering chamber 60. The volume of the metering chamber 60 determines the desired amount of pharmaceutical composition that is to be administered. Metering volumes, for example, range from twenty-five microliters to a hundred microliters. How the metering chamber 60 fills is explained in more detail below. In any event, in that first position of the valve stem 52 no pharmaceutical composition can escape from the metering chamber 60 to the outside, since the metering chamber 60 is sealed from the outside by the stem gasket 64.

In the second position of the valve stem 52, the spring F is compressed and the valve stem 52 is pushed so far into the interior 58 of the valve housing 54 that there is no communication from the interior 58 of the valve housing 54 and from the cavity of the canister 10 via the first channel 66. In that second position of the valve stem 52, there is communication from the metering chamber 60 out to the user by means of a second transverse bore 72 at the “inner” end of the second channel 68. The amount of pharmaceutical composition disposed in the metering chamber 60 can expand through that second transverse bore 72 and the second channel 68 and thus be administered to the user either directly or by means of an adapter, i.e. an oral mouthpiece (not shown).

When the valve stem 52 is released again after the administration, the second transverse bore 72 passes into the region of the stem gasket 64, and the metering chamber 60 is sealed from the outside again. The valve stem 52 is at that point not yet back in its first end position, but the transverse bore 70 is already in communication with the cavity 32 of the canister 10, and as a result of the pressure difference (excess pressure in the canister cavity, discharged metering chamber), the pharmaceutical composition immediately flows from the cavity 32 of the canister 10 filling into the metering chamber 60. The metering chamber 60 is thus immediately refilled when the valve stem 52 is released or returned and the next administration can therefore follow immediately.

Materials for use in the manufacture of the metering chamber 60 and/or the valve stem 52 are known in the prior art and to one of ordinary skill in the art. Examples of suitable materials for the gaskets and seals include, but are not limited to, thermoplasts, elastomers (e.g., neoprene, isobutylene, isoprene, butyl rubber, nitrile rubber); terpolymers of ethylene, propylene and a diene (e.g., butadiene); and fluorinated polymers. The other elements of the metering chamber 60 can be made of corrosion resistant metals (and/or alloys thereof) and/or a plastic.

As used herein, the term “pharmaceutical composition” means a solution or suspension comprising a therapeutic compound (e.g., in the form of solid or liquid particles) to be administered to a mammal, e.g., a human, in a liquid propellant; a mixture of a liquid propellant and a solvent; or an aqueous vehicle. A pharmaceutical composition is “pharmaceutically acceptable” which refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

As used herein, the term “therapeutic compound” means any compound, substance, drug, medicament or active ingredient having a therapeutic or pharmacological effect, and which is suitable for administration to a mammal, e.g., a human. Such therapeutic compounds should be administered in a “therapeutically effective amount”.

As used herein, the term “therapeutically effective amount” refers to an amount or concentration which is effective in reducing, eliminating, treating, preventing or controlling the symptoms of a disease or condition affecting a mammal. The term “controlling” is intended to refer to all processes wherein there may be a slowing, interrupting, arresting or stopping of the progression of the diseases and conditions affecting the mammal. However, “controlling” does not necessarily indicate a total elimination of all disease and condition symptoms, and is intended to include prophylactic treatment.

The therapeutic compound(s) is present in the pharmaceutical compositions of the present invention in a therapeutically effective amount or concentration. Such a therapeutically effective amount or concentration is known to one of ordinary skill in the art as the amount or concentration varies with the therapeutic compound being used and the indication which is being addressed. For example, in accordance with the present invention, the final therapeutic compound concentration in the pharmaceutical composition is, for example, between 0.005% to 10% by weight of the composition; e.g., 0.01% to 1% by weight of the composition. The concentration, for example, will be such as to deliver a therapeutically effective amount of the medicament in one or two actuations of the metering valve.

Therapeutic compounds that are particularly suited for the present invention are those that are thermally labile, for example, at or above room temperature. As used herein, the term “thermally labile” refers to a compound that is susceptible to physical, chemical, biological or microbiological changes during storage. The term thermally labile compounds also includes compounds that are likely to influence the quality, safety and/or efficacy of other therapeutic compounds, for example, formoterol fumarate, salmererol xinafoate, fluticason propionate or proteins.

The therapeutic compound, for example, is in particulate form of a mass median diameters so as to permit inhalation into the bronchial airways which is generally less than a hundred microns; e.g., from about one to about ten microns; e.g., from about one to about five microns.

Examples of therapeutic classes of therapeutic compounds include, but are not limited to, analgesics, anesthetics, scabicides, pediculicides, antineoplastics, antiperspirants, antipruritics, antipsoriatic agents, antiseborrheic agents, antihypertensives, antianxiety agents, anticlotting agents, anticonvulsants, blood glucose-lowering agents, decongestants, antihistamines, antitussives, antineoplastics, beta (β)-blockers, anti-inflammatories, sunscreens, wound healing agents, antipsychotic agents, cognitive enhancers, anti-atherosclerotic agents, cholesterol reducing agents, antiobesity agents, autoimmune disorder agents, anti-impotence agents, antibacterial and antifungal agents, hypnotic agents, cauterizing agents, cleansing agents, deodorants, depigmenting agents, photosensitizing agents, hair growth stimulants, keratolytics, acne agents, antibiotics, anti-depressants, anti-Parkinsonism agents, anti-Alzheimer's disease agents, antiviral agents and combinations of the foregoing.

Especially useful therapeutic compounds for use in the present invention are those materials capable of being formulated into another formulation for administration to the respiratory system (including the nose) and skin. For example, a therapeutic compound in accordance with the present invention could be administered so that it is absorbed into the bloodstream through the lungs. Moreover, the therapeutic compound can be a powdered drug which is effective to treat some condition of the lungs or respiratory system directly and/or topically. Examples of such therapeutic compounds include, but are not limited to, corticosteroids, e.g., mometasone furoate, ciclesonide, beclomethasone dipropionate, budesonide, fluticasone, dexamethasone, flunisolide, triamcinolone, (22R)-6α,9α-difluoro-11β,21-dihydroxyl-16α,17α-propylmethylenedioxy-4-pregnen-3,20-dione, tipredane and the like; β-agonists (i.e. β1 and/or β2-agonists), e.g., salbutamol, albuterol, terbutaline, bitolterol, formoterol, bambuterol, fenoterol, clenbuterol, procateroo, and broxaterol; anticholinergics, e.g., ipratropium bromide, oxitropium bromide, sodium cromoglycate, nedrocromil sodium; leukotriene antagonists, e.g., zafirlukast, prankilast.

Inhalable proteins or peptides can also be suitable for use in the present invention, for example, insulin, interferons, calcitonins, parathyroid hormones, granulocyte colony-stimulating factors, etc.

The final therapeutic compound concentration in the pharmaceutical composition is, for example, between 0.005% to 10% by weight of the composition; e.g., 0.01% to 1% by weight of the composition. The concentration, for example, will be such as to deliver a therapeutically effective amount of the medicament in one or two actuations of the metering valve.

As used herein, the term “propellant” refers to a pharmacologically inert liquid with boiling points from about room temperature to about −25° C. which singly or in combination exerts a high vapor pressure at room temperature. Examples of propellants include, but are not limited to, fluorohydrocarbons (e.g., tetrafluoroethane or heptafluoropropane); hydrocarbons (e.g., butane, propane); and compressed gases.

In addition to the therapeutic compound and the propellant, the pharmaceutical composition can optionally comprise pharmaceutically acceptable excipients. Examples of excipients include, but are not limited to, surfactants, stabilizers, preservatives, dispersing agents; flavorants, anti-oxidants, anti-aggregating agents, and co-solvents.

The insulated canister of the present invention can be filled with the pharmaceutical composition using techniques as known in the art; for example, dual stage pressure filing, single stage cold filling and single stage pressure filling.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

What is claimed:

1. A canister suitable for use in a metered dose delivery system comprising: an outer container, an inner container shaped to be receivable within said outer container to define a gap between said inner container and said outer container, wherein said gap is closed; and a metering valve mounted on said canister.

2. The canister of claim 1, wherein said inner container defines a chamber that is suitable for filling with a pharmaceutical composition.

3. The canister of claim 2, wherein said chamber of said inner container has a surface coating compatible with a pharmaceutical composition.

4. The canister of claim 1, wherein said gap is evacuated and has a negative pressure.

5. The canister of claim 4, wherein said negative pressure is from about 400 mbars to about 800 mbars.

6. The canister of claim 1, wherein said gap is occupied by material having a low thermal conductivity.

7. The canister of claim 6, wherein said low thermal conductivity ranges from about 0.0001 to 0.5 W·m⁻¹·K⁻¹.

8. The canister of claim 1, wherein said gap is filled with a gas.

9. The canister of claim 8, wherein said gas is air.

10. A metered dose delivery system comprising:
(a) a canister comprising an outer container, an inner container shaped to be receivable within said outer container to define a gap between said inner container and said outer container, wherein said gap is closed; and a metering valve mounted on said canister; and
(b) a composition contained in said canister, wherein said composition comprises a therapeutically effective amount of a therapeutic compound and a propellant.

11. The metered dose delivery system of claim 10, wherein said therapeutic compound is a thermally labile therapeutic compound.

12. The metered dose delivery system of claim 10, wherein said therapeutic compound is a respiratory therapeutic compound for inhalation.

13. The metered dose delivery system of claim 10, wherein said gap is evacuated and has a negative pressure.

14. The metered dose delivery system of claim 13, wherein said negative pressure is from about 400 mbars to about 800 mbars.

15. The metered dose delivery system of claim 10, wherein said gap is occupied by material having a low thermal conductivity.

16. The metered dose delivery system of claim 15, wherein said low thermal conductivity ranges from about 0.0001 to 0.5 W·m⁻¹·K⁻¹.

17. The metered dose delivery system of claim 10, wherein said gap is filled with a gas.

18. The metered dose delivery system of claim 17, wherein said gas is air.

19. The metered dose delivery system of claim 10, wherein said therapeutic compound is a dermatological therapeutic compound for topical administration.
